(19) United States
(12) Patent Application Publication
(10) Pub. No.: US 2010/0049251 A1
(43) Pub. Date: Feb. 25, 2010

(54) METHOD AND DEVICE FOR INTERSPINOUS PROCESS FUSION

(76) Inventors: Stephen D. Kuslich, Stillwater, MN (US); Karen Roche, Stillwater, MN (US); Patricia Kocian Kuslich, Stillwater, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/414,250

(22) Filed: Mar. 30, 2009

Related U.S. Application Data
(60) Provisional application No. 61/040,477, filed on Mar. 28, 2008.

(51) Int. Cl. A61B 17/70; A61B 17/88

(52) U.S. Cl. 606/249; 606/279

(57) ABSTRACT

The device of the invention includes a flexible container that may be filled with fill material either before or after placement at the insertion site. It is one object of the present invention to provide a flexible container filled with morselized cortico-cancellous bone graft. According to one aspect of the present invention, a filled flexible container may be placed at the insertion site and then molded to the local anatomy. In an embodiment, a filled flexible container may be placed between two spinous processes.

### RELATED APPLICATION

This application claims the benefit of U.S. Provisional Application No. 61/040,477 filed Mar. 28, 2008, which is hereby fully incorporated herein by reference.

### FIELD OF THE INVENTION

This invention relates to methods and devices for fusing joints, in particular for fusing adjacent spinous processes.

### BACKGROUND OF THE INVENTION

In instances of spondylolisthesis, laminal defects, degenerative spinal stenosis, facet syndrome, fracture or other disease or defect of the spine or other joints, it may be desirable to stabilize the area by affecting fusion of the involved segment. Several devices exist for performing a interspinous fusion. Most include two salient features, the ability to distract the spinous process to a more anatomically normal position relieving pressure on the nerve roots and to restrict extension motion to improve local stability. Currently available devices are usually categorized as static devices or dynamic devices. Static devices are designed to maintain a constant degree of distraction between the spinous processes. However, because the lumbar spine is immobile, when a static device is used the degree of distraction varies with flexion and extension. For example, in extension the fit of the device may be tighter and in tension the fit of the device may be looser.

One method of statically treating the interspinous process includes using an “H”-shaped bone graft. The “H” graft generally consists of a flat section of bone from the tibia or ilium. The ends of the graft are notched to receive the spinous processes at each end of the fusion area. Beyond the variable distraction issues, static devices like an “H” graft have several other disadvantages, including their tendency to dislodge or dislocate from the treatment site, mechanical failure, and static devices often erode the healthy bone adjacent the device leading to device related osteoporosis and/or accelerated degeneration at the adjacent bone.

Dynamic devices have been developed to overcome the disadvantage of the variance in distraction resulting from static devices. Dynamic devices such as the Coflex from Paradigm Spine, may be inserted in a compressed form, the device then expands or distracts with flexion. Another type of dynamic device consists of elastomeric materials that behave as cushion-like materials between the spinous processes. Dynamic devices also have several disadvantages, including requiring a large surgical exposure for insertion into the treatment site, the tendency for the device to produce wear debris and the fact that dynamic devices tend to act merely as spacers and generally do not promote fusion.

There is a need for a device that encompasses the stability of a static device and the motion preservation of a dynamic device.

### SUMMARY OF THE INVENTION

The device of the invention includes a flexible container that may be filled with fill material either before or after placement at the insertion site. It is one object of the present invention to provide a flexible container filled with morselized cortico-cancellous bone graft. According to one aspect of the present invention, a filled flexible container may be placed at the insertion site and then molded to the local anatomy. In an embodiment, a filled flexible container may be placed between two spinous processes.

In one embodiment, a spinous tension band may be placed around the spinous processes to maintain the proximity of the flexible container to the spinous processes. It is another object of the invention to minimize graft migration out of the treatment site. It is yet another object of the invention to maintain the graft in place during the healing process at the treatment site.

According to one aspect of the present invention, the flexible container may be porous such that bone and other cells may migrate from the host treatment site through the container and into the graft material. It is an object of the invention that new bone will grow across the flexible container and that the flexible container filled with graft or other suitable material is strong enough to hold the position of the graft or other material during the healing process.

It is another object of the invention that the graft, or other fill material within the flexible container, bears the compressive forces naturally transmitted through the joint, whereas the container itself experiences tensile force while maintaining the graft or other material in the desired position to promote healing.

It is yet another object of the present invention to treat an interspinous process by implanting a flexible container filled with graft or other suitable material into a treatment site such that the foramen is opened increasing the foraminal volume and maintaining the increased foraminal volume.

It still another object of the present invention to treat an interspinous process by implanting a flexible container into a treatment site and then filling the container with graft or other suitable material such that the foramen is opened increasing the foraminal volume and maintaining the increased foraminal volume.

It is another object of the present invention to mechanically stabilize the affected joint while reducing the stiffness in the affected joint by providing a flexible container filled with fill material that remains in position and is able to incorporate new bone growth to promote healing.

### BRIEF DESCRIPTION OF THE DRAWINGS

FIG. 1 depicts an embodiment of the present invention placed in a thoracic interspinous process.

FIG. 2 depicts an embodiment of the present invention placed in a cervical interspinous process.

FIG. 3 is a side view of an embodiment of the present invention placed in a lumbar interspinous process.

FIG. 4 is an axial view of an embodiment of the present invention placed in a lumbar interspinous process.

FIG. 5 is a posterior view of an embodiment of the present invention placed in a lumber interspinous process.

FIG. 6 depicts the compressive and tensile forces imparted on an embodiment of the present invention.

### DETAILED DESCRIPTION OF THE INVENTION

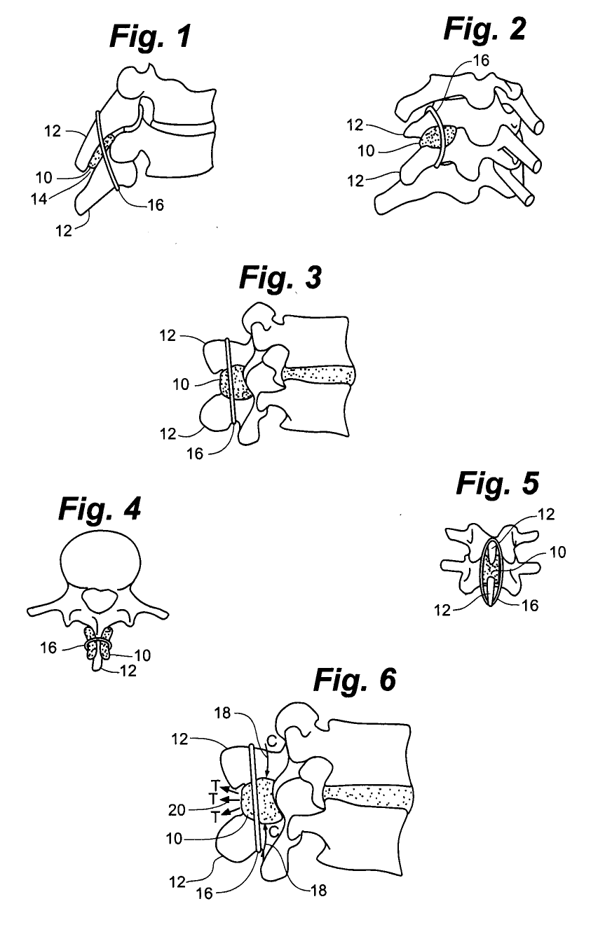

According to one embodiment of the invention as depicted in FIG. 1-5, device 10 may be a flexible container such that device 10 may conform to the anatomy of the treatment site. In an embodiment, device 10 may be an elliptical shape, an “H” shape or any other shape to conform to the desired anatomy. Device 10 may be used to treat damaged, diseased or otherwise abnormal joints including, but not limited to, interspinous processes, wrists and ankles. According to one aspect, device 10 may be comprised of material including, but not limited to polyurethane, nylon, polypropylene, nitonol, thread or woven material, any suitable plastic, any suitable polymer or any other suitable material or combination thereof.

By way of example, device 10 will be described with respect to treating the interspinous space 12. One of skill in the art will readily recognize that device 10 is not limited to treating an interspinous space, but may be used to treat other joints as well. In a preferred embodiment, device 10 may be a flexible porous container, one example of which is described in U.S. Pat. No. 7,226,481, which is hereby fully incorporated herein by reference. Device 10 may be made of material that is woven, knitted, braided or form-molded to a density that will allow ingress and egress of fluids and solutions and will allow interdigitation, that is, the ingrowth and through-growth of blood vessels and fibrous tissue and bony trabeculae, but the fabric porosity is tight enough to retain small particles of enclosed material, such as for example, ground up bone graft, or bone graft substitute such as hydroxyapatite or other osteoconductive biocompatible materials known to promote bone formation.

Generally, the pores 14 of device 10 may have a diameter generally in the range of about 0.25 mm or less to about 5.0 mm. The pore size is selected to allow tissue ingrowth while containing the material packed into the bag. If bone cement or other material is used which will not experience bone ingrowth, pores 14 may be much tighter to prevent egress of fill material from within device 10 out into the surrounding anatomy. This prevents fill material from exiting the treatment site and that possibly impinging upon nerves, blood vessels or other sensitive anatomy.

Suitable fill material may be one or more of the following, or any other biocompatible material judged to have the desired physiologic response: A) Demineralized bone material, morselized bone graft, cortical, cancellous, or cortico-cancellous, including autograft, allograft, or xenograft; B) Any bone graft substitute or combination of bone graft substitutes, or combinations of bone graft and bone graft substitutes, or bone inducing substances, including but not limited to: Tricalcium phosphates, Tricalcium sulfates, Tricalcium carbonates, hydroxyapatite, bone morphogenic protein, calcified and/or decalcified bone derivative; and C) Bone cements, such as ceramic and polymethylmethacrylate bone cements.

In an embodiment the pore size may be generally in range of about 500-2500 microns and the fill material may include a combination of materials, one example of such a combination is described in co-pending U.S. Application Publication No. 2005/0131417 A1, which is hereby fully incorporated herein by reference. In one embodiment, such combination may include granules of an osteoconductive material and an osteoinductive material carried in a lubricating carrier.

The lubricating carrier may generally be a viscous liquid, for example, sodium hyaluronan in varying molecular weights, alginate, dextran, gelatin, collagen and others. According to one embodiment, the osteoinductive material may be non-demineralized cortical cancellous allograft granules or other suitable osteoconductive material, which may be fully contained by device 10 due to their physical size, and can thereby provide some structural strength to the joint. The granules provide a focus for load bearing or load sharing just as the pebbles in concrete. The ratio of cortical to cancellous allograft may be in the range of 25:75-100:0.

The granules may be mixed with demineralized bone matrix allograft (“DBM”) or other suitable osteoinductive material, which is a fine particulate. As device 10 is filled with the cortical cancellous allograft granules, some of the particulate DBM may be retained within the filled device 10, but a portion of it may be free to flow out through pores 14 of device 10. This results in a surrounding “halo” of osteoinductive material at the margins of filled device 10 in direct apposition with the surrounding host tissue where it can initiate recruitment of the stem cells, thus encouraging bone growth to heal the joint.

In an embodiment of device 10 as depicted in FIG. 5, device 10 may be configured in a generally “H” shape. According to one aspect, “H” shaped device 10 may be filled with fill material prior to insertion. In an embodiment, device 10 may be filled with morselized cortico-cancellous bone graft. Morselized graft has been shown to incorporate with local host bone faster than traditional cortical bone blocks. In one embodiment, filled device 10 may then be placed between the interspinous processes 12. Because device 10 is flexible and conformable, once device 10 is in place, device 10 may be conformed to the local anatomy. When treating interspinous processes 14, device 10 is filled to a sufficient volume and positioned such that the foramen is opened increasing the foraminal volume. Device 10 is further positioned to maintain the increased foraminal volume. A tension band 16 may then be placed around the spinous processes 14 such that the proximity of device 10 to spinous processes 14 is maintained. Embodiments of a tension band 16 useful with the presentation are described, for example, in U.S. Pat. Nos. 6,656,185 and 6,695,852, the disclosure of which is hereby incorporated by reference.

According to one aspect of the invention, device 10 contains the fill material and thus prevents undesired migration of fill material into the surrounding anatomy while maintaining the fill material in the desired position to promote healing. Animal testing of a porous embodiment of device 10, shows that cells from the host tissue will migrate from the host and into the fill material, that new bone will grow across device 10. Further, as shown in FIG. 6, the fill material feels the compression forces 18 naturally transmitted through the joint and device 10 only experiences tensile forces 20. This force distribution construct gives device 10 the strength to hold the position of the fill material under compression between the spinous processes during the healing process.

The placement of device 10 at both sides of the interspinous process increases the range of motion in the affected joint while reducing the stiffness in the affected joint by providing a flexible container filled with fill material that remains in position and is able to incorporate new bone growth to promote healing.

This completes the description of the preferred and alternate embodiments of the invention. Those skilled in the art may recognize other equivalents to the specific embodiment described herein which equivalents are intended to be encompassed by the claims attached hereto.

1. A system for treating a spinal motion segment including adjacent spinous processes, the spinal motion segment having a first diminished foraminal volume comprising:
a conformable container adapted for placement between the adjacent spinous processes;
a fill tool that introduces fill material into the container such that the fill material applies a force within the container sufficient to increase the first diminished foraminal volume to a second increased foraminal volume and maintain the second increased foraminal volume.

2. The system of claim 1 further wherein the container is porous.

3. The system of claim 1 further wherein the container is nonporous.

4. The system of claim 1 wherein the container is configured in an H shape.

5. A system for treating a spinal motion segment including adjacent spinous processes comprising:
a conformable container adapted for placement between the adjacent spinous processes;
a fill tool that introduces fill material into the container such that the filled container mechanically stabilizes the spinal motion segment.

6. The system of claim 5 further wherein the container is porous.

7. The system of claim 5 further wherein the container is nonporous.

8. The system of claim 5 wherein the container is configured in an H shape.

9. A system for treating a spinal motion segment including adjacent spinous processes comprising:
a conformable container adapted for placement between the adjacent spinous processes;
a fill tool that introduces fill material into the container such that the filled container remains placed between the adjacent spinous processes to incorporate new bone growth and promote healing.

10. The system of claim 9 further wherein the container is porous.

11. The system of claim 9 further wherein the container is nonporous.

12. The system of claim 9 wherein the container is configured in an H shape.

13. A method of treating a spinal motion segment including adjacent spinous processes, the spinal motion segment having a first diminished foraminal volume comprising the steps of:
placing a conformable container between the adjacent spinous processes;
introducing fill material into the container such that the fill material applies a force within the container sufficient to increase the first diminished foraminal volume to a second increased foraminal volume and maintains the second increased foraminal volume.

14. A method of treating a spinal motion segment including adjacent spinous processes comprising:
placing a conformable container between the adjacent spinous processes;
introducing fill material into the container such that the filled container mechanically stabilizes the spinal motion segment.

15. A method of treating a spinal motion segment including adjacent spinous processes comprising:
placing a conformable container between the adjacent spinous processes;
introducing fill material into the container such that the filled container remains placed between the adjacent spinous processes to incorporate new bone growth and promote healing.

16. A method of treating a spinal motion segment including adjacent spinous processes, the spinal motion segment having a first diminished foraminal volume comprising:
providing a system comprising a conformable container adapted for placement between the adjacent spinous processes and a fill tool that introduces fill material into the container;
providing instructions for using the system including the steps of:
placing the conformable container between the adjacent spinous processes;
introducing the fill material into the container such that the fill material applies a force within the container sufficient to increase the first diminished foraminal volume to a second increased foraminal volume and maintains the second increased foraminal volume.

17. A method of treating a spinal motion segment including adjacent spinous processes comprising:
providing a system comprising a conformable container adapted for placement between the adjacent spinous processes and a fill tool that introduces fill material into the container;
providing instructions for using the system including the steps of:
placing the conformable container between the adjacent spinous processes;
introducing fill material into the container such that the filled container mechanically stabilizes the spinal motion segment.

18. A method of treating a spinal motion segment including adjacent spinous processes comprising:
providing a system comprising a conformable container adapted for placement between the adjacent spinous processes and a fill tool that introduces fill material into the container;
providing instructions for using the system including the steps of:
placing the conformable container between the adjacent spinous processes;
introducing fill material into the container such that the filled container remains placed between the adjacent spinous processes to incorporate new bone growth and promote healing.
